(19) United States
(12) Patent Application Publication
(10) Pub. No.: US 2012/0279508 A1
(43) Pub. Date: Nov. 8, 2012

(54) Restraint

(76) Inventors: Leanne Marie McBean, Edmonton (CA); Blaise Junior McPherson, Edmonton (CA)

(21) Appl. No.: 13/067,014

(22) Filed: May 3, 2011

(51) Int. Cl. A61F 5/37

(52) U.S. Cl. 128/876; 128/870

(57) ABSTRACT

An apparatus for supporting a user comprising a continuous unitary elongate flexible member having first and second surfaces. The elongate member extends between first and second ends and has a central portion therebetween. The apparatus further comprises a pad disposed along the first surface of the central portion. The method comprises locating a central portion of the elongate member behind the neck of the user such that the pad is dispose between the elongate member and the neck of the user. The method further comprises passing the first and second ends of the elongate member over the shoulders and under the arms of the user such that the first and second ends are located behind the back of the user and securing the first and second ends to an object so as to secure and support the torso of the user thereto

### BACKGROUND OF THE INVENTION

1. Field of Invention

The present invention relates to straps in general and in particular to a rapid and convenient method and apparatus for supporting the torso of a user.

2. Description of Related Art

In the medical field it is frequently necessary to support or restrain a portion of the body of a patient. In particular, some patients, either during or shortly after receiving treatment may become disoriented or incapable of supporting themselves. For such patients it is frequently necessary to ensure that they do not injure themselves by falling or otherwise bumping into other people or objects.

At other times, some patients may become agitated due to stress, trauma, drug interactions or mental illness. For such patients it is also necessary to restrain or otherwise ensure that the patient is not able to injure themselves or others due to impacts or falls. For such patients it is also necessary to ensure that they are not able to leave the hospital or other facility until they can be appropriately treated.

In many of these situations, it is frequently necessary for the care worker or security personnel to have rapid access to a restraint or support device. Previous attempts to provide such a device for securing or otherwise supporting the body of a patient have been unsatisfactory. Such previous attempts have included large buckles which are bulky and limit the ability of the device to be folded and carried with the care worker or security personnel. Such buckles may also be disadvantageously be utilized as improvised weapons by uncooperative patients. Examples of such devices are shown in U.S. Pat. Nos. 5,526,824 to McAllister and 6,202,647 to Espino.

Additionally, some of the previous attempts to provide such a device have included multiple straps and harness members. Such devices however, in additional to being bulky and therefore less prone to being carried around by a care worker or security personnel may also be time consuming and difficult to apply to the patient in need of such a device. Examples of such devices are shown in U.S. Pat. Nos. 3,137,294 to Robertson and 3,191,599 to Kendell.

### SUMMARY OF THE INVENTION

According to a first embodiment of the present invention there is disclosed an apparatus for supporting a user comprising a unitary elongate flexible member having first and second surfaces. The elongate member extends between first and second ends and has a central portion therebetween. The apparatus further comprises a pad disposed along the first surface of the central portion wherein the elongate member has a length sufficient to be located around the back of the neck of a user such that the first and second ends are passed over the users shoulders and under their armpits so as to be connectable behind the back of the user.

The elongate member may comprise a strap. The strap may be substantially non-elastic. The strap may be formed of a material selected from the group consisting of cotton, nylon and leather. The elongate member may have a length of between 6 and 14 feet. The elongate member may have a width of between 1 and 2 inches.

The pad may be permanently secured to the elongate member. The pad may be sewn to the elongate member. The pad may have a width of between 2 and 4 inches. The pad may have a length of between 3 and 6 feet. The pad may have a thickness of between ⅜ of an inch and 1 inch. The pad may be formed of a material selected from the group consisting of cotton, polyester batting, foam and felt.

According to a further embodiment of the present invention there is disclosed a method of supporting the torso of a user comprising locating a central portion of an elongate member behind the neck of the user such that a pad secured to the elongate member is dispose between the elongate member and the neck of the user. The method further comprises passing the first and second ends of the elongate member over the shoulders and under the arms of the user such that the first and second ends are located behind the back of the user and securing the first and second ends to an object so as to secure and support the torso of the user thereto

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

In drawings which illustrate embodiments of the invention wherein similar characters of reference denote corresponding parts in each view,

FIG. 1 is a perspective view of an apparatus for supporting or restraining a user according to a first embodiment of the present invention.

FIG. 2 is a cross sectional view of the apparatus of FIG. 1 as taken along the line 2-2.

FIG. 3 is a perspective view of the apparatus of FIG. 1 in a folded or stored position.

FIG. 4 is a perspective view of the apparatus of FIG. 1 being applied to a user in a first position.

FIG. 5 is a perspective view of the apparatus of FIG. 1 applied to a user in a second position.

### DETAILED DESCRIPTION

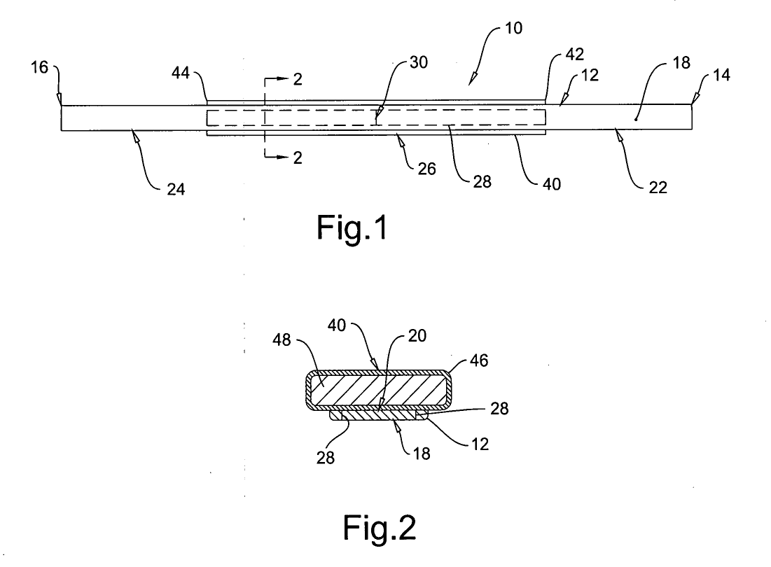

Referring to FIGS. 1 and 2, an apparatus for supporting or restraining a user according to a first embodiment of the invention is shown generally at 10. The apparatus comprises an elongate strap 12 having a pad 40 applied thereto. The strap 12 extends between first and second free distal ends, 14 and 16, respectively and includes front and rear surfaces, 18 and 20, as illustrated in FIG. 2. The strap 12 includes first and second end portions, 22 and 24, respectively and a central portion 26 to which the pad 40 is secured. In operation, the first and second end portions, 22 and 24 may be tied to each other or an adjacent object with the central portion 26 and pad 40 supporting the wearer as will be further described below.

The pad 40 comprises a rectangular body extending between first and second ends, 42 and 44 respectively. The pad 40 is secured to the rear surface 20 of the central portion 26 of the strap and is selected have a width greater than the width of the strap. The pad 40 is secured to the strap 12 by stitching or other suitable means indicated generally at 28. As illustrated in FIG. 1, the pad 40 is centred on the strap by stitching 28 although it will be appreciated that other securing means may be utilized as well, such as, by way of non-limiting example, adhesives. As illustrated in FIG. 1, the stitching 28 may extend along the length of the pad 40 to either side of the strap 12 and an optional center mark 30 may also be stitched therein so as to indicate the midpoint of the strap and pad. Optionally, a differently colored line or other type of visual indicator may be utilised to provide the center mark 30.

The strap 12 may be formed of any suitable non-elastic material, such as, by way of non-limiting example, twill, cotton, nylon or leather. In some embodiments, the material should also be selected to be fast and simple to tie a knot in and also to hold the knot without excessive slippage thereof. The strap 12 may be formed to have a thickness of between 1/16 and ⅛ inch (2 and 3 mm) although it will be appreciated that other thicknesses may be utilized as well depending upon the material selected so as to provide adequate padding to a wearer. The strap 12 has a width selected to provide sufficient strength in supporting or restraining the torsos of a user while being as comfortable as possible for the wearer and being light weight and foldable for transportation by the care provider or security personnel. In practice it has been found that a width of between 1 and 2 inches (25 and 51 mm) has been useful. The length of the strap 12 is selected to permit it to be extended around the patient in need of support as will be further described below. In practice, it has been found that a length of between 6 and 14 feet (1.8 and 4.2 meters) has been useful depending upon the size and age of the intended patient. In particular a length of 10 feet (3.0 meters) has been found to be particularly useful for adult patients.

The pad 40 may be formed of any suitable material, such as by way of non-limiting example, cotton, polyester batting, foam and felt. In some embodiments, a sheath layer 46 may be included over a padding layer 48 as illustrated in FIG. 2 so as to provide a softer material for contact with the patient's skin so as to increase patient comfort by reducing itching from the batting material, to more evenly distributing pressure from the apparatus as well as to provide a more pleasing appearance of the apparatus. It will also be appreciated that in some embodiments, such as, by way of non-limiting example when a thick felt batting pad 40 is utilized that a sheath layer 46 may not be necessary. In one particular embodiment, the sheath layer 46 may be formed of a porous elasticized fabric whereas the padding layer 48 is formed of a polyester blend quilt batting.

The pad 40 may be formed to have a thickness of between ⅜ and 1 inch. (10 to 25 mm). The pad 40 has a width selected to adequately distribute the force exerted by the apparatus on the patient when in use restraining the torsos of a user and to not form any pressure points which will cause the wearer discomfort. The pad 40 should also be thin and light weight enough to be foldable for transportation by the care provider or security personnel. In addition, the width of the pad 40 will be selected to aid in preventing the strap 12 from twisting when applied to a wearer. In practice it has been found that a width of between 2 and 4 inches (51 and 102 mm) has been useful. The length of the pad 40 is selected to bear against the user in all points of contact and pressure so as to prevent the narrower strap 12 from unduly applying a greater surface pressure to the user. In practice, it has been found that a length of between 3 and 6 feet (0.9 and 1.8 meters) has been useful depending upon the size and age of the intended patient. In particular a length of 4 feet (1.2 meters) has been found to be particularly useful for adult patients.

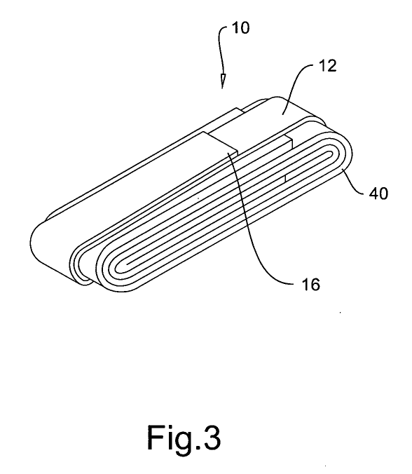

Turning now to FIG. 3, the apparatus 10 is illustrated in a folded or stored position. In such a position, the apparatus may be folded and/or wrapped around itself in successive rotations until one free distal end, such as the second end 16 is remaining which may thereafter be located proximate to the previous winding. In such a configuration, the apparatus may be easily stored and transported within a pouch, pocket or the like of a care worker or security personnel.

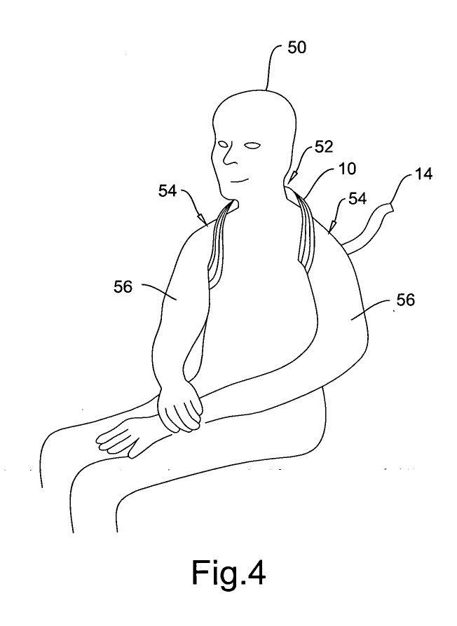

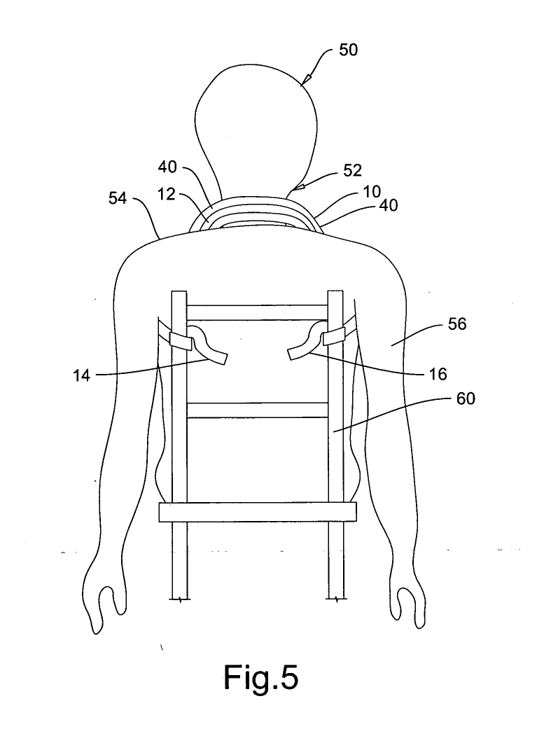

With reference to FIGS. 4 and 5, in operation, the apparatus 10 may be located behind the neck 52 of a wearer 50 with the pad 40 adjacent to the wearer's neck 52. Preferably, the center mark 30 of the strap may be centred on the wearer's neck such that an equal length of the strap extends to either side thereof. Thereafter, the first and second ends 14 and 16 of the strap may be passed over the shoulders 54 of the user and under their arms 56 so as to terminate behind the back of the user as illustrated with reference to the first end 14 in FIG. 4. As illustrated in FIG. 5, the first and second ends 14 and 16 may be either secured to an object behind the wearer, such as, by way of non-limiting example, a chair 60 hospital bed, or wheelchair or wrapped around the object and tied to each other to the object to secure the torso of the wearer thereto. Optionally, the apparatus may be wrapped around the torso of the wearer and the first and second ends 14 and 16 may be tied to each other across the chest of the wearer for situations where the patient is lying down and it was undesirable to have them sit up or for use in tying and securing the wrists and ankles of the patient. In other applications, the apparatus may be included in a first aid kit for use as an impromptu bandage, for use in tying splints or for replacing a triangular bandage, by way of non-limiting example.

While specific embodiments of the invention have been described and illustrated, such embodiments should be considered illustrative of the invention only and not as limiting the invention as construed in accordance with the accompanying claims.

What is claimed is:

1. An apparatus for supporting a user comprising:
a unitary elongate flexible member having first and second surfaces, said elongate member extending between first and second ends and having a central portion therebetween; and
a pad disposed along said first surface of said central portion,
said elongate member having a length sufficient to be locatable around the back of the neck of a user such that said first and second ends are passed over the users shoulders and under their armpits so as to be securable behind the back of the user.

2. The apparatus of claim 1 wherein said elongate member comprises a strap.

3. The apparatus of claim 2 wherein said strap is substantially non-elastic.

4. The apparatus of claim 3 wherein said strap is formed of a material selected from the group consisting of cotton, nylon and leather.

5. The apparatus of claim 1 wherein said elongate member has a length of between 6 and 14 feet.

6. The apparatus of claim 1 wherein said strap has a width of between 1 and 2 inches.

7. The apparatus of claim 1 wherein said pad is permanently secured to said elongate member.

8. The apparatus of claim 7 wherein said pad is sewn to said elongate member.

9. The apparatus of claim 1 wherein said pad has a width of between 2 and 4 inches.

10. The apparatus of claim 1 wherein said pad has a length of between 3 and 6 feet.

11. The apparatus of claim 1 wherein said pad has a thickness of between ⅜ of an inch and 1 inch.

12. The apparatus of claim 1 wherein said pad is formed of a material selected from the group consisting of cotton, polyester batting, foam and felt.

13. A method of supporting the torso of a user, the method comprising:
locating a central portion of an elongate member behind the neck of said user such that a pad secured to said elongate member is dispose between said elongate member and said neck of the user;
passing said first and second ends of said elongate member over the shoulders and under the arms of the user such that said first and second ends are located behind the back of the user; and
securing said first and second ends to an object so as to secure and support the torso of the user thereto
